(19) United States
(12) Patent Application Publication
(10) Pub. No.: US 2010/0119443 A1
(43) Pub. Date: May 13, 2010

(54) RADIOTHERAPEUTIC SYSTEM AND METHOD PERFORMED BY NON-INVASIVE AND REAL-TIME TUMOR POSITION TRACKING

(75) Inventors: Young Hoon JI, Seoul (KR); Hai Jo JUNG, Namyangju-si (KR); Kum Bae KIM, Seoul (KR); Seung Woo PARK, Seoul (KR)

Correspondence Address: Jason Y. Pahng and Associates, LLC, 12178 Bridgend Run, Fairfax, VA, 22030, US

(73) Assignee: KOREA INSTITUTE OF RADIOLOGICAL & MEDICAL SCIENCES, Seoul (KR)

(21) Appl. No.: 12/614,444

(22) Filed: Nov. 8, 2009

(30) Foreign Application Priority Data
Nov. 11, 2008 (KR) 10-2009-0111866

(51) Int. Cl. A61K 51/00; A61B 6/00

(52) U.S. Cl. 424/1.11; 600/436

(57) ABSTRACT

A radiotherapeutic system including non-invasive and real-time tumor tracking includes: a signal detector for detecting radioactive rays emitted from a tumor to which the radioactive medical supply is adsorbed and generating an electrical signal; a signal processor for converting the electrical signal generated into a three-dimensional (3D) coordinate signal; a controller for generating a control signal in conjunction with the 3D coordinate signal output by the signal processor; and a radioactive-ray irradiation device for emitting radioactive rays to the tumor, wherein the amount of the radioactive rays is controlled by the controller.

### CROSS-REFERENCE TO RELATED PATENT APPLICATION

This application claims the benefit of Korean Patent Application No. 10-2008-0111866, filed on Nov. 11, 2008, in the Korean Intellectual Property Office, the disclosure of which is incorporated herein in its entirety by reference.

### BACKGROUND OF THE INVENTION

1. Field of the Invention

The present invention relates to a radiotherapeutic system and method of precisely irradiating radioactive rays to a tumor site of moving internal organs while tracking the tumor site in real time when radiotherapy is performed.

The present invention is as a result of research performed as part of the atomic power research and development project of the Ministry of Education, Science, and Technology [Project Number: M20702000001-08N0200-00110, Project title: Medical and Physics Technique Development for Radiotherapy while Tracking Movement of Internal Organs Radioactive Rays]

2. Description of the Related Art

Modern people living in complicated societies are under a great deal of stress and have irregular eating habits. Thus, it is more difficult to stay in good shape. In particular, vicious tumors, that is, cancer is the leading cause of death for modern people. In today's society, the number of cancer cases is increasing and thus national-scale solutions to cope with the increasing trend are urgently is needed. Thus, various cancer therapies, in particular, radiotherapy are receiving more attention as possible solutions.

In general, tumor radiotherapy is performed to kill a tumor (or cancer) alone by intensely irradiating radioactive rays only to a tumor site while minimizing irradiation to normal tissues surrounding the tumor.

However, according to conventional radiotherapy, radioactive rays are irradiated to the entire moving site to kill the tumor of moving internal organs. However, this method has low therapeutic efficiency and also leads to the destruction of normal cells.

Meanwhile, the movement of a target site can be preciously tracked by inserting metal or a RF radio frequency coil into the body by surgery. This method, however, causes additional pain and discomfort to patients.

These problems may also be solved by indirectly tracking the movement of a tumor while therapy is performed, where the indirect tracking method includes labeling the tissue of a target tumor with a label, photographing the label using a camera, and tracking the movement of the obtained image of the label.

However, this method also has a low degree of precision because the tumor site is indirectly tracked, not directly tracked.

### SUMMARY OF THE INVENTION

The present invention provides a radiotherapeutic system and method of irradiating radioactive rays to a tumor while detecting relatively strong radioactive rays emitted from a tumor through which radioactive medical supplies(radiopharmaceutical), which have been used only for diagnosis, are administered and adsorbed to a patient are adsorbed and tracking the position of the tumor in real time.

According to an aspect of the present invention, there is provided a radiotherapeutic system including non-invasive and real-time tumor tracking, wherein the radiotherapeutic system includes: a signal detector for detecting radioactive rays emitted from a tumor to which the radioactive medical supply is adsorbed and generating an electrical signal; a signal processor for converting the generated electrical signal into a three-dimensional (3D) coordinate signal; a controller for generating a control signal in conjunction with the 3D coordinate signal output by the signal processor; and a radioactive-ray irradiation device for emitting radioactive rays to the tumor, wherein the amount of the radioactive rays is controlled by the controller.

The signal detector may include at least two gamma-ray detectors that are disposed to be perpendicular to each other near the tumor.

The radioactive-ray irradiation device may include a multileaf collimator device for emitting radioactive rays in an amount that is continuously controlled by the controller.

The multileaf collimator device may include a body that is movable according to the position of the tumor.

The radioactive-ray irradiation device may include a switch that is intermittently opened or closed by the controller to irradiate radioactive rays.

According to another aspect of the present invention, there is provided a radiotherapeutic method including: injecting a radioactive medical supply to a human body; emitting radioactive rays by a tumor to which the radioactive rays are adsorbed; detecting radioactive rays emitted from the tumor; tracking the position of the tumor using the detected radioactive rays; and irradiating radioactive rays to the tumor while tracking the position of the tumor in real time.

### BRIEF DESCRIPTION OF THE DRAWINGS

The above and other features and advantages of the present invention will become more apparent by describing in detail exemplary embodiments thereof with reference to the attached drawings in which:

FIG. 1 is a schematic view of a radiotherapeutic system including non-invasive and real-time tumor tracking, according to an embodiment of the present invention;

FIG. 2 is a schematic diagram for explaining a signal detector;

FIGS. 3 through 5 are diagrams for explaining a radioactive-ray irradiation device; and

FIG. 6 is a flowchart of a radiotherapeutic method performed by non-invasive and real-time tumor tracking, according to an embodiment of the present invention.

### DETAILED DESCRIPTION OF THE INVENTION

Hereinafter, embodiments of the present invention will be described in detail with reference to the attached drawings.

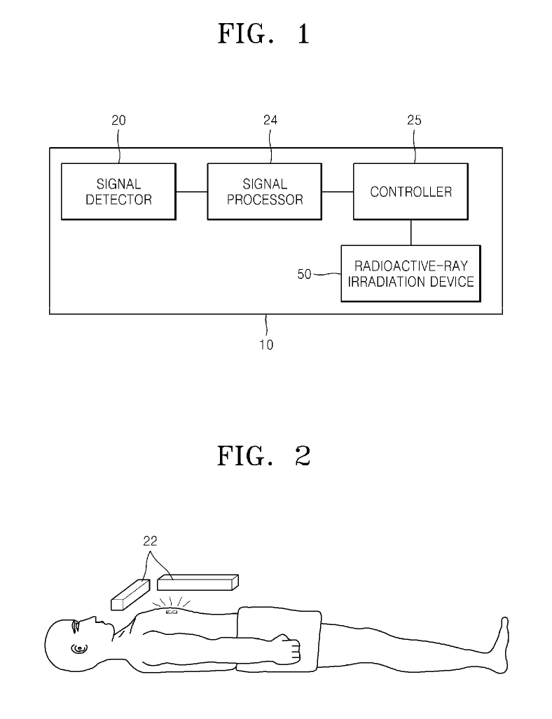

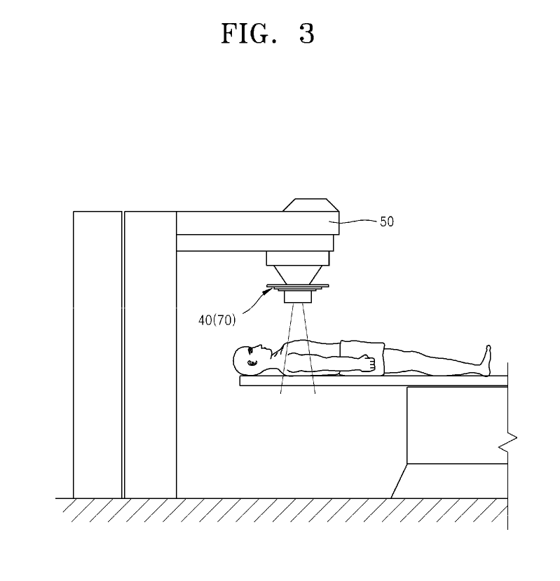

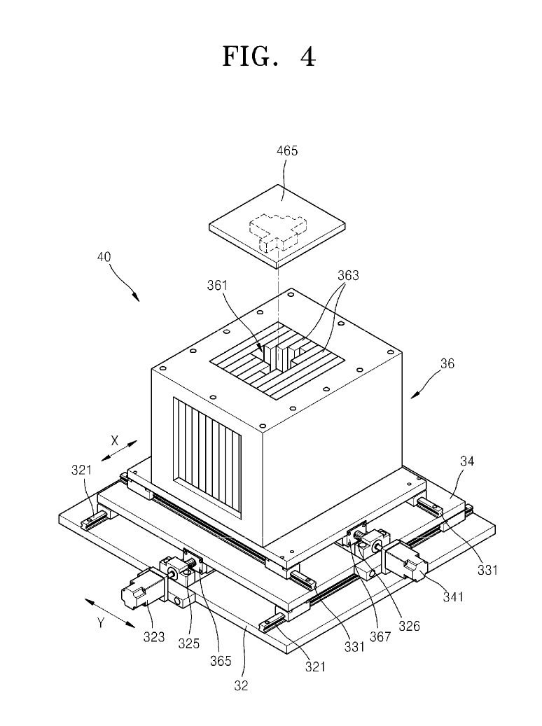

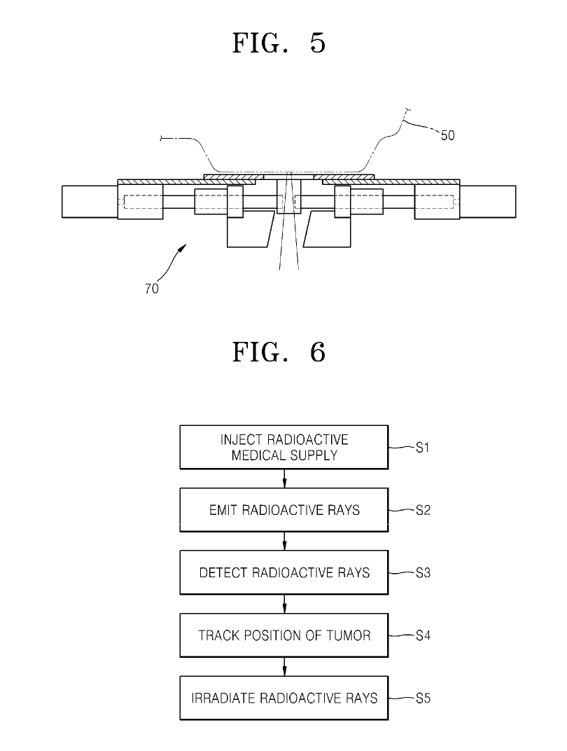

FIG. 1 is a schematic view of a radiotherapeutic system 10 including non-invasive and real-time tumor tracking, according to an embodiment of the present invention, FIG. 2 is a schematic diagram for explaining a signal detector 20, FIGS. 3 through 5 are diagrams for explaining a radioactive-ray irradiation device 50, and FIG. 6 is a flowchart of a radiotherapeutic method performed by non-invasive and real-time tumor tracking, according to an embodiment of the present invention.

Referring to FIGS. 1 through 6, the radiotherapeutic system 10 including non-invasive and real-time tumor tracking, according to the present embodiment (hereinafter, referred to as “radiotherapeutic system”), includes the signal detector 20, a signal processor 24, a controller 25, and the radioactive-ray irradiation device 50.

In order to operate the radiotherapeutic system 10, a radioactive medical supply needs to be injected to a human body. The radioactive medical supply is selectively adsorbed to a tumor site in the human body and emits radioactive rays. Since more radioactive medical supply is adsorbed more to a tumor site than to normal cells, the tumor site emits more radioactive rays than normal cells. Examples of the radioactive medical supply include 2-[18F]fluoro-2-deoxy-D-glucose (FDG) and L-[11C-methyl]methionine that is useful for diagnosing cancer, in particular, a brain tumor. Meanwhile, examples of an isotope for proton emission tomography include 18F, 11C, 15O, and 13N.

The signal detector 20 detects radioactive rays emitted from a tumor to which the radioactive medical supply is adsorbed and generates an electrical signal. The signal detector 20 may include, for example, a gamma-ray detector 22. The gamma-ray detector 22 detects radioactive rays emitted from a tumor to which the radioactive medical supply is adsorbed and generates an electrical signal. If the gamma-ray detector 22 has high resolution, a detection time for detecting radioactive rays emitted from the tumor is long. On the other hand, if the gamma-ray detector 22 has low resolution, the detection time for detecting radioactive rays emitted from the tumor is short. Since the objective of the present invention includes irradiating radioactive rays to a tumor while tracking a position of the tumor in real time, the resolution of the gamma-ray detector 22 may be controlled to be as low as possible. Thus, when the resolution of the gamma-ray detector 22 is lowered to a predetermined value or less, radioactive rays emitted from the tumor are detected in real time. Thus, the signal processor 24, which will be described later, calculates a three-dimensional (3D) position of the tumor using the electrical signal generated by the signal detector 20. When two or more gamma-ray detectors 22 are used, the gamma-ray detectors 22 are disposed perpendicular to each other near the tumor. Each of the gamma-ray detectors 22 includes a plurality of gamma-ray detection cells that are arranged on the same plane. A gamma-ray detector 22 detects a two-dimensional position of the tumor. Thus, when two gamma-ray detectors 22 are placed perpendicular to each other and the generated electrical signals are mathematically processed, three-dimensional coordinates of the tumor are obtained.

The signal processor 24 converts the electrical signal generated by the signal detector 20 into a digital signal by using an analog-to-digital converter (ADC), and then mathematically processes the digital signal to obtain the 3D position of the tumor and outputs the obtained 3D position of the tumor. The signal processor 24 tracks the strongest signal among signals detected by the signal detector 20. The signal processor 24 generates a signal indicating the image of the tumor obtained by Anger Logic, which is a conventionally known image process algorithm. Since the reproduction of an image by converting an analog signal (electrical signal) input to the signal processor 24 into a digital signal is easily performed using a known signal process technique, detailed description thereof will not be presented herein. The position of the tumor may be tracked in real time by tracking a position of a spot emitting the strongest signal in images generated by the signal processor 24.

The controller 25 controls the radioactive-ray irradiation device 50 according to the position of the tumor, which is calculated and processed by the signal processor 24 so that radioactive rays are irradiated to the tumor while the radioactive-ray irradiation device 50 tracks the tumor in real time. That is, the controller 25 generates a control signal in conjunction with a coordinate signal output by the signal processor 24. Since the controller 25 is easily embodied with a known electronical engineering technique, the detailed description thereof will not be presented herein.

The irradiation dose of radioactive rays irradiated to the tumor by the radioactive-ray irradiation device 50 is controlled by the controller 25. The radioactive-ray irradiation device 50 irradiates therapeutic radioactive rays to a target site in a patient. The radioactive-ray irradiation device 50 is a device that generates radioactive rays by accelerating electrons or particles and irradiates the generated radioactive rays, which is well known in physics and medical fields. Thus, the structure and principal of the radioactive-ray irradiation device 50 will not be described in detail.

The radioactive-ray irradiation device 50 includes a control device that directly controls irradiation of radioactive rays to the tumor. The control device may be a multileaf collimator device 40 illustrated in FIG. 4 or a switch 70 illustrated in FIG. 5. Referring to FIG. 4, the multileaf collimator device 40 may include a multileaf collimator 363 that includes individual ‘leaves’ each having a flat form and a radioactive-ray permeable portion having the same shape as the target site. The multileaf collimator device 40 is installed in the radioactive-ray irradiation device 50 such that the multileaf collimator device 40 relatively moves with respect to the radioactive-ray irradiation device 50. That is, the movement of the multileaf collimator device 40 is controlled by a servo motor, as will be described later, and a control signal for controlling the servo motor is transmitted by the controller 25.

FIG. 3 illustrates the radioactive-ray irradiation device 50 and the multileaf collimator device 40 installed in the radioactive-ray irradiation device 50. FIG. 4 illustrates the multileaf collimator device 40 in more detail than in FIG. 3. Referring to FIG. 4, the multileaf collimator device 40 includes a body 32, a sliding member 34, a frame 36, a first servo motor 323, and a second servo motor 341.

The body 32 is fixed with respect to the radioactive-ray irradiation device 50. The body 32 includes a first through-hole (not shown). The first through-hole is disposed in a pathway of high-energy radioactive rays that are accelerated and moved toward the target site in the radioactive-ray irradiation device 50. Two guide rails 321 are mounted on the body 32. The body 32 may include metal such as carbon steel or aluminum alloys. However, the body 32 may also include materials other than metal, and may include any material that allows the body 32 to support the frame 36 as will be described later. The first servo motor 323 may be installed on the body 32.

The sliding member 34 is installed such that the sliding member 34 is movable with respect to the body 32 in one direction. The sliding member 34 is, as illustrated in FIG. 4, installed such that the sliding member 34 is able to slide with respect to the body 32 along the guide rail 321 on the body 32 in a first direction X. The sliding member 34 may include a second through-hole (not shown) corresponding to the first through-hole of the body 32. In the present embodiment, the sliding member 34 is connected to the first servo motor 323 by a ball screw 325 and a ball nut 365, which are widely used to transform rotational motion to linear motion, such that the first servo motor 323 provides power to the sliding member 34. That is, the ball screw 325 is fixed to an output shaft of the first servo motor 323, and the ball nut 365, which is screw-coupled to the ball screw 325, is fixed to the sliding member 34. Thus, when the first servo motor 323 rotates, the ball screw 325 rotates and thus, the sliding member 34 fixed to the ball nut 365 slides in the first direction X. Meanwhile, instead of the ball screw 325 and the ball nut 365, a rack and a pinion may also be used.

The second servo motor 341 is disposed to be perpendicular to the first servo motor 323 and is fixed to the sliding member 34.

The frame 36 is, as illustrated in FIG. 4, installed such that the frame 36 is able to slide with respect to the sliding member 34 along guide rails 331 in a second direction Y. The frame 36 may include a through-hole 361 through which radioactive rays irradiated by the radioactive-ray irradiation device 50 pass. The through-hole 361 may be located to correspond to the first through-hole. The through-hole 361 is defined by the multileaf collimator 363. The multileaf collimator 363 is constructed using individual leaves that are able to slide with respect to each other, thereby forming a corrugated structure. Also, the multileaf collimator 363 is able to slide with respect to the frame 36. The frame 36 has an open surface so that an end of the multileaf collimator 363 is pushed or pulled. The multileaf collimator 363 may include carbon steel or a tungsten alloy, each of which is capable of shielding radioactive rays so that the radioactive rays irradiated by the radioactive-ray irradiation device 50 are allowed or not allowed to pass through according to need. The multileaf collimator 363 may be manually manipulated. Meanwhile, the multileaf collimator device 40 may further include a template 465 for setting the shape of a region through which radioactive rays pass and which is determined by the shape of the opening of the multileaf collimator 363. The template 465 may include an acrylic material. Various templates 465 having shapes corresponding to the shape of the target site of a patient may be manufactured in advance. In a state in which the multileaf collimator 363 is manipulated to have the through-hole 361, the template 465 is disposed on the through-hole 361 and then, a sliding manipulation is performed such that the multileaf collimator 363 only opens a portion of the through-hole 361 corresponding to the template 465 while shielding the other portion of the through-hole 361. Thus, radioactive rays pass through in the shape of the template 465 corresponding to the target site of a patient.

The first direction X is perpendicular to the second direction Y. Thus, the frame 36 is disposed such that the frame 36 two-dimensionally moves on the sliding member 34 with respect to the body 32. The frame 36 is connected to the second servo motor 341 such that the second servo motor 341 provides power to the frame 36, and according to the present embodiment, like the sliding member 34 and the first servo motor 323, the frame 36 is connected to the second servo motor 341 by a ball screw 326 and a ball nut 367 such that the second servo motor 341 provides power to the frame 36.

The controller 25 generates a signal for controlling driving of the first servo motor 323 and the second servo motor 341. The controller 25 is electrically connected to the first servo motor 323 and the second servo motor 341 through an electrical wire. The controller 25 receives from the signal processor 24 position data according to the movement of a tumor and then based on the position data, generates a signal for controlling driving of the first servo motor 323 and the second servo motor 341 so that radioactive rays are continuously irradiated to the target site while the multileaf collimator 363 follows the tumor. Thus, in the multileaf collimator device 40, the body 36 moves according to the position of the tumor.

Meanwhile, like an opening and closing driver disclosed in Korean Registration Patent Publication No. 0740430, the switch 70 illustrated in FIG. 5 may be used instead of the multileaf collimator device 40. Referring to Korean Registration Patent Publication No. 0740430, the switch 70 is installed in the radioactive-ray irradiation device 50 in such a manner as illustrated in FIG. 5 so that the switch 70 is intermittently opened or closed by the controller 25 to irradiate radioactive rays.

As described above, the radioactive-ray irradiation device 50 includes the multileaf collimator device 40 or the switch 70, each of which controls the irradiation dose of radioactive rays irradiated to the tumor by the radioactive-ray irradiation device 50.

As described above, in the radiotherapeutic system 10, a radioactive medical supply injected in a human body is adsorbed to a tumor, radioactive rays emitted from the tumor are detected by the signal detector 20, and then the signal processor 24, the controller 25, and the radioactive-ray irradiation device 50 sequentially operate to precisely irradiate radioactive rays to the tumor. In particular, the radiotherapeutic system 10 is characterized in that a tumor is treated while the position of the tumor is tracked in real time. In addition, unlike conventional tumor tracking methods, the radiotherapeutic system 10 uses non-invasive tumor tracking without additional pain and discomfort.

A tumor radiotherapy using the radiotherapeutic system 10 including non-invasive and real-time tumor tracking will be described.

The tumor radiotherapy sequentially includes a first operation (S1), a second operation (S2), a third operation (S3), a fourth operation (S4), and a fifth operation (S5).

In the first operation (S1), a radioactive medical supply is provided to the body of a patient by injection.

In the second operation (S2), the radioactive medical supply is adsorbed to a tumor and radioactive rays are emitted therefrom. From the time when radioactive rays are emitted, the radiotherapeutic system 10 is used. In the third operation (S3), the signal processor 24 of the radiotherapeutic system 10 detects radioactive rays emitted from the tumor. The gamma-ray detectors 22 installed in the signal detector 20 are disposed to be perpendicular to each other and generate an electrical signal for calculating 3D position data of the tumor.

In the fourth operation (S4), the position of the tumor is tracked using the radioactive rays detected in the third operation (S3). That is, the signal processor 24 converts an electrical signal generated by the signal detector 20 into a digital signal by using an ADC, and then mathematically processes the digital signal to obtain 3D position data of the tumor. As described above, the connection between the signal detector 20 and the signal processor 24 enables real-time tracking of the position of the tumor. In the fifth operation (S5), while tracking the position of the tumor obtained in the fourth operation (S4), radioactive rays are irradiated to the tumor in real time. In the fifth operation (S5), an accurate irradiation dose of radioactive rays are irradiated to the tumor while the multileaf collimator device 40 of the radioactive-ray irradiation device 50 moves according to the position of the tumor in response to a control signal of the controller 25.

As described above, a radiotherapeutic system and method according to embodiments of the present invention provide tumor radiotherapy that is performed while tracking the position of a tumor in the body of a patient in real time without additional pain and discomfort.

Although according to the present embodiment, the signal detector 20 includes two gamma-ray detectors disposed to be perpendicular to each other near the tumor, three or more gamma-ray detectors may also be used.

Although according to the present embodiment, the radioactive-ray irradiation device 50 includes the multileaf collimator device 40 that through the controller 25 continuously controls the irradiation dose of radioactive rays, other devices, such as the switch 70 illustrated in FIG. 5, which controls the radiation dose of radioactive rays may also be used instead of the multileaf collimator device 40.

Although according to the present embodiment, the multileaf collimator device 40 includes the body 32 that is movable according to the position of the tumor, a bed on which a patient lies is moved while the body 32 is fixed.

A radiotherapeutic system and method which are performed by non-invasive and real-time tumor tracking, according to embodiments of the present invention, are non-invasive so that a patient does not suffer from additional pain and discomfort, and uses real-time tracking according to the position of the tumor. In addition, according to the radiotherapeutic system and method, the position of the tumor is directly tracked in real time and thus radiotherapy can be precisely performed on the tumor.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A radiotherapeutic system comprising non-invasive and real-time tumor tracking, the radiotherapeutic system comprising:
a signal detector for detecting radioactive rays emitted from a tumor to which the radioactive medical supply is adsorbed and generating an electrical signal;
a signal processor for converting the generated electrical signal into a three-dimensional (3D) coordinate signal;
a controller for generating a control signal in conjunction with the 3D coordinate signal output by the signal processor; and
a radioactive-ray irradiation device for emitting radioactive rays to the tumor, wherein the amount of the radioactive rays is controlled by the controller.

2. The radiotherapeutic system of claim 1, wherein the signal detector comprises at least two gamma-ray detectors that are disposed to be perpendicular to each other near the tumor.

3. The radiotherapeutic system of claim 1, wherein the radioactive-ray irradiation device comprises a multileaf collimator device for emitting radioactive rays in an amount that is continuously controlled by the controller.

4. The radiotherapeutic system of claim 3, wherein the multileaf collimator device comprises a body that is movable according to the position of the tumor.

5. The radiotherapeutic system of claim 1, wherein the radioactive-ray irradiation device comprises a switch that is intermittently opened or closed by the controller to irradiate radioactive rays.

6. A radiotherapeutic method comprising:
injecting a radioactive medical supply to a human body;
emitting radioactive rays by a tumor to which the radioactive rays are adsorbed;
detecting radioactive rays emitted from the tumor;
tracking the position of the tumor using the detected radioactive rays; and
irradiating radioactive rays to the tumor while tracking the position of the tumor in real time.
